(19) United States
(12) Patent Application Publication
(10) Pub. No.: US 2017/0192067 A1
(43) Pub. Date: Jul. 6, 2017

(54) SYSTEMS AND METHODS FOR HEAT MANAGEMENT IN A MAGNETIC RESONANCE IMAGING SYSTEM

(71) Applicant: General Electric Company, Schenectady, NY (US)

(72) Inventors: DANIEL GARCIA, PEWAUKEE, WI (US); CHINMOY GOSWAMI, WAUKESHA, WI (US); JASON MONTCLAIR PITTMAN, WAUKESHA, WI (US); AMY SUE MEYERS, MADISON, WI (US)

(73) Assignee: General Electric Company, Schenectady, NY (US)

(21) Appl. No.: 14/987,020

(22) Filed: Jan. 4, 2016

(51) Int. Cl. G01R 33/34; G01R 33/28; A61B 5/055

(52) U.S. Cl. CPC G01R 33/3403; G01R 33/34092; A61B 5/055; G01R 33/288

(57) ABSTRACT

A radio frequency coil includes a body having an inner wall and an outer wall opposite the inner wall. The body is configured to fit over an imaging bore of a magnetic resonance imaging system such that the inner wall is closer to the imaging bore than the outer wall. The body may have a cooling duct embedded in the body between the inner wall and the outer wall and configured to direct a coolant to at least one assembly component disposed in the magnetic resonance imaging system. The cooling duct may be formed by the body. A phase change material may be disposed on the body or embedded in the body between the inner wall and the outer wall. The phase change material may be configured to absorb heat emitted by at least one assembly component of the magnetic resonance imaging system.

### BACKGROUND

Technical Field

Embodiments of the invention relate generally to management of heat in a magnetic resonance imaging system (MRI).

Discussion of Art

MRI is a widely accepted and commercially available technique for obtaining digitized visual images representing the internal structure of objects having substantial populations of atomic nuclei that are susceptible to nuclear magnetic resonance (NMR). Many MRI systems use magnet assemblies that house superconductive magnets to impose a strong main magnetic field on the nuclei in the patient/object to be imaged within a target volume (herein after also referred to as the “imaging bore”). The nuclei are excited by a radio frequency (RF) signal, typically emitted via a RF coil, at characteristics NMR (Larmor) frequencies. By spatially disturbing localized magnetic fields surrounding the object within the imaging bore, and analyzing the resulting RF responses from the nuclei as the excited protons relax back to their lower energy normal state, a map or image of these nuclei responses as a function of their spatial location is generated and displayed. An image of the nuclei responses provides a non-invasive view of an object's internal structure.

Many magnet assemblies have components, sometimes referred to as “hot components” (herein after also referred to as “assembly components”) that emit significant amounts of heat during operation/imaging of the MRI. For example, many magnet assemblies include processors, electro-magnetic coils and/or other electrically conductive assembly components that emit heat when powered by an electrical current. In particular, many superconductive magnets generate strong magnetic fields by manipulating (i.e., switching amplitude, frequencies, direction, etc.) an electrical current within a gradient coil. The level and/or rate of manipulation of the electrical current within a gradient coil is known as “gradient performance.” Manipulation of the electrical current within the gradient coil, however, causes the gradient coil to emit heat. As a result, the amount of heat emitted by a gradient coil typically increases with increased gradient performance.

The heat emitted by the assembly components of an MRI system is potentially hazardous to the magnetic assembly and/or other components of the MRI system. For example, typical magnetic assemblies include electrical processors and other integrated circuits, as well as soldered connections, which may melt and/or burn at high temperatures. Additionally, the RF coils used in many magnet assemblies typically conduct heat emitted by assembly components towards the imaging bore, thereby raising the temperature of the imaging bore. Current regulations limit the temperature of the imaging bore to a maximum of 41° C.

Accordingly, once the imaging bore temperature reaches a threshold temperature, many MRI systems must cease operations (herein after also referred to as being “rested”) in order to allow the imaging bore time to cool and return to a lower temperature. Such resting, however, limits the number of MRI images that can be taken within a given time period. Thus, the cost-effectiveness of many MRI systems is reduced by the problems associated with heat emitted by assembly components.

Moreover, aggressive MRI imaging/scanning (e.g., high resolution, small field of view (“FOV”), knee, wrist and/or spine imaging), tends to require high levels of gradient performance. As such, aggressive MRI imaging often increases the amount heat emitted by a gradient coil, which in turn shortens the amount of operational time between MRI resting periods and further reduces the cost-effectiveness of an MRI system. The demand for aggressive MRI imaging is increasing, however.

What is needed, therefore, is a system and method to better manage heat within a magnetic resonance imaging system.

### BRIEF DESCRIPTION

In an embodiment, a radio frequency coil is provided. The radio frequency coil includes a body and a cooling duct. The body has an inner wall and an outer wall opposite the inner wall. The body is configured to fit over an imaging bore of a magnetic resonance imaging system such that the inner wall is closer to the imaging bore than the outer wall. The cooling duct is embedded in the body between the inner wall and the outer wall and configured to direct a coolant to at least one assembly component disposed in the magnetic resonance imaging system. The cooling duct is formed by the body.

In another embodiment, another radio frequency coil is provided. The radio frequency coil includes a body and a phase change material. The body has an inner wall and an outer wall opposite the inner wall. The body is configured to fit over an imaging bore of a magnetic resonance imaging system such that the inner wall is closer to the imaging bore than the outer wall. The phase change material is configured to absorb heat emitted by at least one assembly component of the magnetic resonance imaging system. The phase change material is disposed on the body or embedded in the body between the inner wall and the outer wall.

In yet another embodiment, a method is provided. The method includes cooling at least one assembly component of a magnetic resonance imaging system via a coolant directed by a cooling duct. The cooling duct is embedded between an inner wall and an outer wall of a body of a radio frequency coil. The outer wall is opposite the inner wall. The body is configured to fit over an imaging bore of a magnetic resonance imaging system such that the inner wall is closer to the imaging bore than the outer wall. The cooling duct is formed by the body.

In yet another embodiment, another method is provided. The method includes absorbing, via a phase change material, heat emitted by at least one assembly component of a magnetic resonance imaging system. The phase change material is disposed on a body of a radio frequency coil or embedded in the body between an inner wall and an outer wall of the body. The outer wall is opposite the inner wall. The body is configured to fit over an imaging bore of the magnetic resonance imaging system such that the inner wall is closer to the imaging bore than the outer wall.

In yet another embodiment, a magnetic resonance imaging system is provided. The system includes at least one assembly component that emits heat, and an imaging bore. A bulk amount of a phase change material is disposed within the magnetic resonance imaging system. The phase change material has a phase transition temperature near an operating temperature of the at least one assembly component such that a rise in a temperature of the imaging bore resulting from heat emitted by at least one assembly component is delayed.

### DRAWINGS

The present invention will be better understood from reading the following description of non-limiting embodiments, with reference to the attached drawings, wherein below:

FIG. 1 is a block diagram of an exemplary MRI system that incorporates embodiments of the invention;

FIG. 2 is a schematic side elevation view of the MRI system of FIG. 1;

FIG. 3 is a perspective view of an exemplary radio frequency coil of the MRI system of FIG. 1 in accordance with embodiments of the invention;

FIG. 4 is another perspective view of the exemplary radio frequency coil of the MRI system of FIG. 1;

FIG. 5 is another perspective view of the exemplary radio frequency coil of the MRI system of FIG. 1;

FIG. 6 is a cutaway perspective view of a body of the radio frequency coil of FIG. 3 in accordance with embodiments of the invention;

FIG. 7 is a schematic side view of a cooling duct embedded within the body of the radio frequency coil of FIG. 3 in accordance with embodiments of the invention; and

FIG. 8 is a graphical model of coolant flowing through cooling ducts embedded within the body of the radio frequency coil of FIG. 3.

### DETAILED DESCRIPTION

Reference will be made below in detail to exemplary embodiments of the invention, examples of which are illustrated in the accompanying drawings. Wherever possible, the same reference characters used throughout the drawings refer to the same or like parts, without duplicative description.

As used herein, the terms “substantially,” “generally,” and “about” indicate conditions within reasonably achievable manufacturing and assembly tolerances, relative to ideal desired conditions suitable for achieving the functional purpose of a component or assembly. As used herein, “electrically coupled, “electrically connected” and “electrical communication” means that the referenced elements are directly or indirectly connected such that an electrical current may flow from one to the other. The connection may include a direct conductive connection (i.e., without an intervening capacitive, inductive or active element), an inductive connection, a capacitive connection, and/or any other suitable electrical connection. Intervening components may be present. The term “phase change material” and/or “PCM” means any material, to include organic and inorganic compounds, that has a high heat of fusion such that it is capable of storing and releasing large amounts of energy. In particular, a PCM may absorb or emit heat and rise or fall, respectively, in temperature until a “transition temperature” is reached. When at the transition temperature, a PCM can absorb or release heat with little or no change in temperature as it transitions between one or more known states of matter (e.g., solid, liquid, gas, plasma, etc.). Some commonly known PCMs are water, sodium sulfate, sodium acetate, lauric acid, TME, aluminum, copper, gold, iron, lead, lithium, silver, titanium, zinc, salt hydrates, and paraffins. Additionally, as used herein, the term “bore temperature” refers to the temperature of a patient/imaging bore of an MRI system. The terms “rest” and “resting,” as used herein, refer to the ceasing of scanning/imaging by a MRI system for the purpose of allowing the bore temperature and/or the temperature of other MRI components to return back to a lower operating temperature. The term “operating temperature” refers to the temperature of a component of a MRI system brought about via scanning/imaging operations conducted by the MRI system. Further, the term “heating time constant” is used to refer to the relationship between the amount of heat emitted by one or more assembly components and the amount of time required to raise the temperature of the imaging bore. For example, the higher the heating time constant, the longer the time and/or the larger the amount of heat needed to raise the temperature of the imaging bore. Further still, the term “assembly component,” as used herein, refers to components of a magnet assembly and/or the encompassing MRI system.

While the embodiments disclosed herein are described with respect to a MRI system, it is to be understood that embodiments of the present invention are equally applicable to devices such as RF cavity-based accelerators, free electron lasers, and any other device that may have assembly components that generate heat. As will be appreciated, embodiments of the present invention related imaging systems may be used to analyze animal tissue generally and are not limited to human tissue.

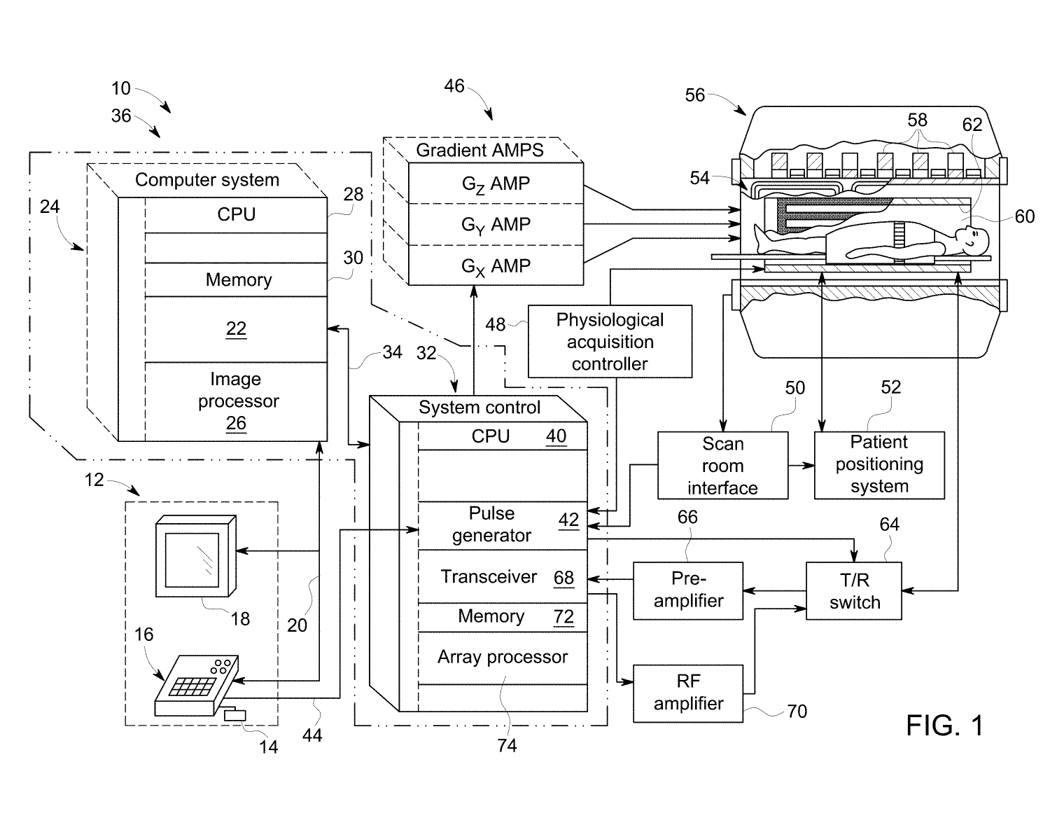

Referring to FIG. 1, the major components of a MRI system 10 incorporating an embodiment of the invention are shown. Operation of the system 10 is controlled from the operator console 12, which includes a keyboard or other input device 14, a control panel 16, and a display screen 18. The console 12 communicates through a link 20 with a separate computer system 22 that enables an operator to control the production and display of images on the display screen 18. The computer system 22 includes a number of modules, which communicate with each other through a backplane 24. These include an image processor module 26, a CPU module 28 and a memory module 30, which may include a frame buffer for storing image data arrays. The computer system 22 communicates with a separate system control or control unit 32 through a high-speed serial link 34. The input device 14 can include a mouse, joystick, keyboard, track ball, touch activated screen, light wand, voice control, or any similar or equivalent input device, and may be used for interactive geometry prescription. The computer system 22 and the MRI system control 32 collectively form an “MRI controller” 36.

The MRI system control 32 includes a set of modules connected together by a backplane 38. These include a CPU module 40 and a pulse generator module 42, which connects to the operator console 12 through a serial link 44. It is through link 44 that the system control 32 receives commands from the operator to indicate the scan sequence that is to be performed. The pulse generator module 42 operates the system components to execute the desired scan sequence and produces data which indicates the timing, strength and shape of the RF pulses produced, and the timing and length of the data acquisition window. The pulse generator module 42 connects to a set of gradient amplifiers 46, to indicate the timing and shape of the gradient pulses that are produced during the scan. The pulse generator module 42 can also receive patient data from a physiological acquisition controller 48 that receives signals from a number of different sensors connected to the patient, such as ECG signals from electrodes attached to the patient. And finally, the pulse generator module 42 connects to a scan room interface circuit 50 which receives signals from various sensors associated with the condition of the patient and the magnet system. It is also through the scan room interface circuit 50 that a patient positioning system 52 receives commands to move the patient to the desired position for the scan.

The pulse generator module 42 operates the gradient amplifiers 46 to achieve desired timing and shape of the gradient pulses that are produced during the scan. The gradient waveforms produced by the pulse generator module 42 are applied to the gradient amplifier system 46 having Gx, Gy, and Gz amplifiers. Each gradient amplifier excites a corresponding physical gradient coil in a gradient coil assembly, generally designated 54, to produce the magnetic field gradients used for spatially encoding acquired signals. The gradient coil assembly 54 forms part of a magnet assembly 56, which also includes a polarizing magnet 58 (which in operation, provides a homogenous longitudinal magnetic field B₀ throughout a target volume 60 that is enclosed by the magnet assembly 56) and a whole-body (transmit and receive) RF coil 62 (which, in operation, provides a transverse magnetic field B₁ that is generally perpendicular to B₀ throughout the target volume 60).

The resulting signals emitted by the excited nuclei in the patient may be sensed by the same RF coil 62 and coupled through the transmit/receive switch 64 to a preamplifier 66. The amplifier MR signals are demodulated, filtered, and digitized in the receiver section of a transceiver 68. The transmit/receive switch 64 is controlled by a signal from the pulse generator module 42 to electrically connect an RF amplifier 70 to the RF coil 62 during the transmit mode and to connect the preamplifier 66 to the RF coil 62 during the receive mode. The transmit/receive switch 64 can also enable a separate RF coil (for example, a surface coil) to be used in either transmit or receive mode.

The MR signals picked up by the RF coil 62 are digitized by the transceiver module 68 and transferred to a memory module 72 in the system control 32. A scan is complete when an array of raw k-space data has been acquired in the memory module 72. This raw k-space data is rearranged into separate k-space data arrays for each image to be reconstructed, and each of these is input to an array processor 74 which operates to Fourier transform the data into an array of image data. This image data is conveyed through the serial link 34 to the computer system 22 where it is stored in memory 30. In response to commands received from the operator console 12, this image data may be archived in long term storage or it may be further processed by the image processor 26 and conveyed to the operator console 12 and presented on the display 18.

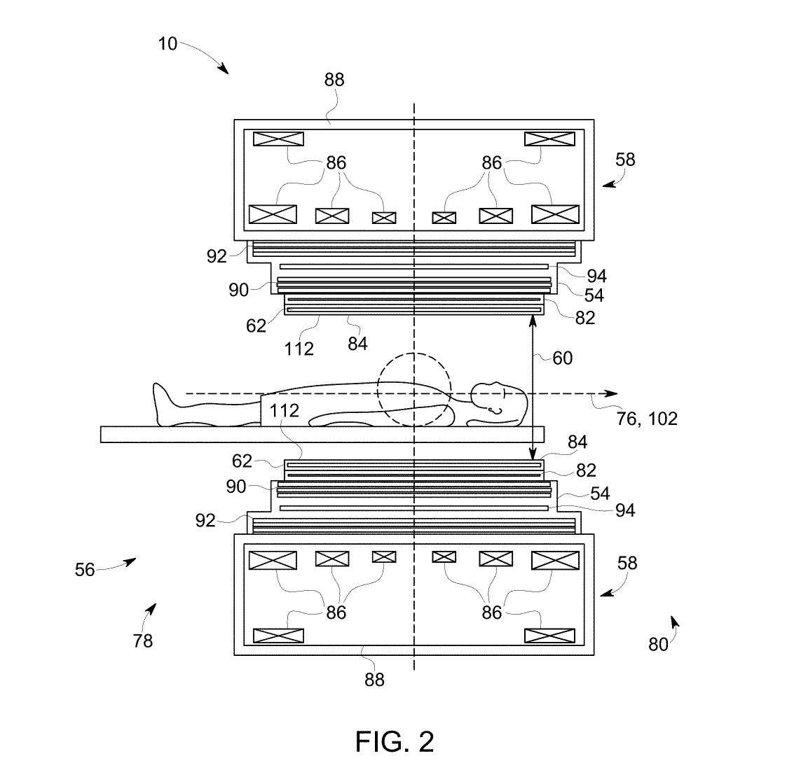

Referring now to FIG. 2, a schematic side elevation view of the magnet assembly 56 is shown in accordance with an embodiment of the invention. The magnet assembly 56 is cylindrical in shape having a center axis 76, a “patient end” 78, and a “service end” 80 opposite of the patient end 78. The magnet assembly 56 includes the polarizing magnet 58, the gradient coil assembly 54, a RF shield 82, the RF coil 62, and an imaging bore 84. The magnetic assembly 56 may further include various other elements such as covers, supports, suspension members, end caps, brackets, etc. which have been omitted from FIG. 2 for clarity. While the embodiment of the magnetic assembly 56 shown in FIGS. 1 and 2 utilize a cylindrical magnet and gradient topology, it should be understood that magnet and gradient topologies other than cylindrical assemblies may be used. For example, a flat gradient geometry in a split-open MRI system may also utilize embodiments of the invention described below.

The polarizing magnetic 58 may include several radially aligned longitudinally spaced apart superconductive coils 86, wherein each coil is capable of carrying a large current. The superconductive coils 86 are designed to create the B₀ field within the patient/target volume 60. The superconductive coils 86 are enclosed in a cryogen environment within a cryostat 88. The cryogenic environment is designed to maintain the temperature of the superconducting coils 86 below the appropriate critical temperature so that the superconducting coils 86 are in a superconducting state with zero resistance. The cryostat 88 may include a helium vessel (not shown) and thermal or cold shields (not shown) for containing and cooling magnet windings in a known manner.

The gradient coil assembly 54 is disposed within the inner circumference of the magnet assembly 56 and around the RF shield 82 and the RF coil 62 in a spaced-apart coaxial relationship. The gradient coil assembly 54 may be mounted to the polarizing magnet 58 such that the gradient coil assembly 54 is circumferentially surrounded by the polarizing magnet 58. The gradient coil assembly 54 may also circumferentially surround the RF shield 82 and the RF coil 62. In embodiments, the gradient coil assembly 54 may be a self-shielded gradient coil assembly. For example, the gradient coil assembly 54 may include a cylindrical inner gradient coil assembly or winding 90 and a cylindrical outer gradient coil assembly or winding 92 both disposed in a concentric arrangement with respect to the center axis 76. The inner gradient coil assembly 90 includes inner (or main) X-, Y- and Z-gradient coils and the outer gradient coil assembly 92 includes the respective outer (or shielding) X-, Y-, and Z-gradient coils. The coils of the inner gradient coil assembly 90 may be activated by passing an electric current through the coils to generate a gradient field in the patient volume 60 as required in MR imaging. A volume 94 or space between inner gradient coil assembly 90 and the outer gradient coil assembly 92 may be filled with a bonding material, e.g., epoxy resin, visco-elastic resin, polyurethane, etc. Alternatively, an epoxy resin with filler material such as glass beads, silica and alumina may be used as the bonding material.

The RF shield 82 is cylindrical in shape and is disposed around the RF coil 62. The RF shield 82 is used to shield the RF coil 62 from external sources of RF radiation and may be fabricated from any suitable conducting material, for example, sheet copper, circuit boards with conducting copper traces, copper mesh, stainless steel mesh, other conducing mesh, etc.

The imaging bore 84 surrounds the cylindrical patient/target volume or bore 60. The imaging bore tube 84 can be configured as a standard bore size (−60 cm) or as a wide bore size (−70 cm or greater). As previously stated, the temperature of the imaging bore 84 may increase during scanning operations due to heat emitted by one or more components of the magnet assembly 56.

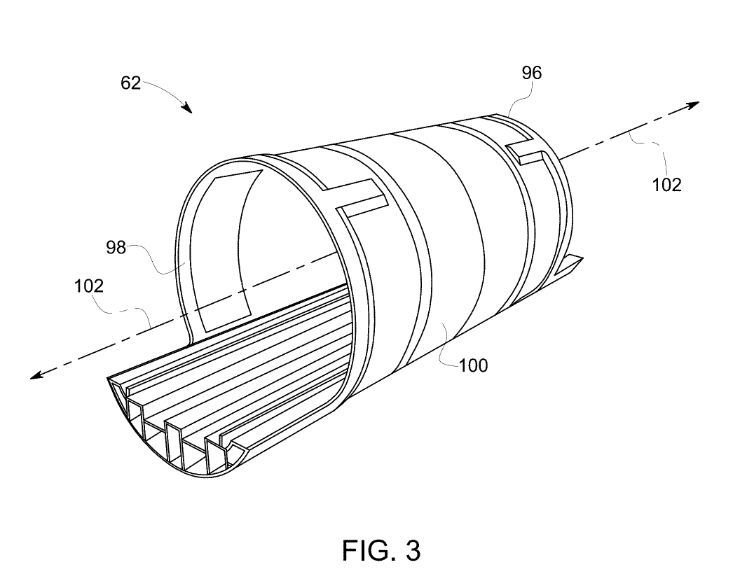

As shown in FIGS. 2 and 3, the RF coil 62 is cylindrical, disposed around an outer surface of the imaging bore tube 84, and may be mounted inside the cylindrical gradient coil assembly 54. The RF coil includes a body 96 having an inner wall 98 and an outer wall 100. The outer wall 100 is disposed opposite the inner wall 98. The body 96 is configured to fit over the imaging bore 84 such that the inner wall 98 is closer to the imaging bore 84 than the outer wall 100. The body includes a longitudinal axis 102 which corresponds to central axis 76.

As previously stated, RF coils 62 tend to absorb heat emitted by other assembly components (e.g., heat emitted by the gradient coil assembly 54 and/or the polarizing magnet 58) within the magnet assembly 56 and conduct said heat towards the imaging bore 84. As also previously stated, such heat can be hazardous to the assembly components and/or the patient/object being imaged within the imaging bore 84. Thus, the present invention seeks to manage the heat within the magnet assembly 56 and/or the encompassing MRI 10 by using the RF coil 62 to locally manage/target heat emitted by individual assembly components. The present invention also seeks to globally manage heat within the magnet assembly 56 and/or the encompassing MRI 10 by removing and/or absorbing heat away from the RF coil 62 which typically would have been conducted by the body 96 of the RF coil 62 towards the imaging bore 84.

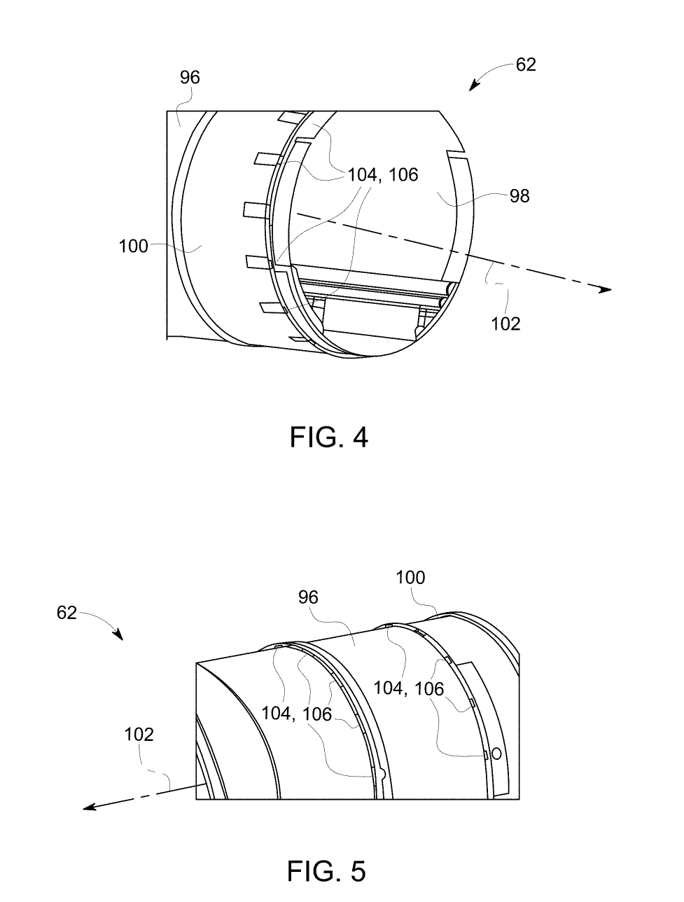

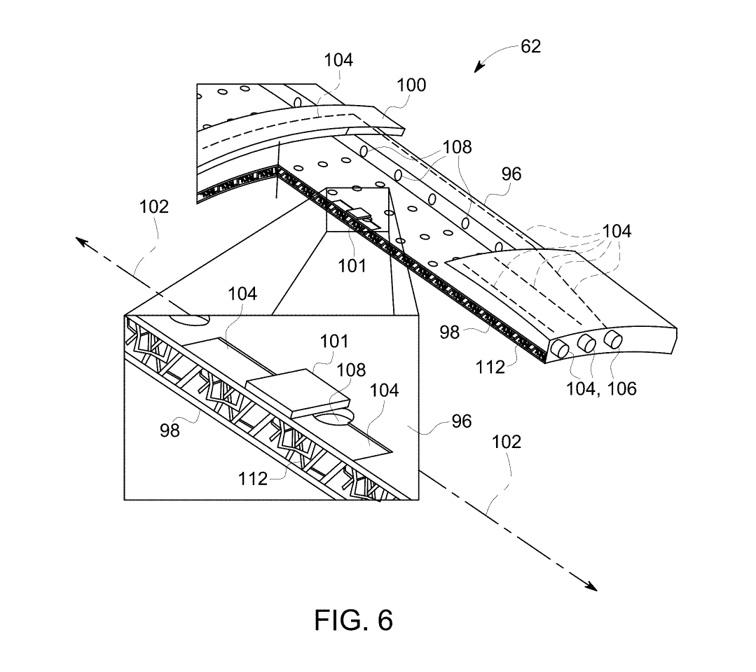

Accordingly, as best seen in FIGS. 4-6, in embodiments, the RF coil 62 further includes one or more cooling ducts 104 embedded in the body 96 between the inner wall 98 and the outer wall 100. The cooling ducts 104 (best shown as dashed lines in FIG. 6) are configured to direct a coolant (e.g., air, water, and/or other types of heat absorbing/transferring substances) to at least one assembly component (e.g. the gradient coil assembly 54, the RF coil 62, the RF shield 82, the imaging bore 84, and/or other components disposed within the magnetic assembly 56 and/or the encompassing MRI 10 such as microprocessors and soldered electrical connections) that emit heat.

The cooling ducts 104 are formed by the body 96. In other words, the cooling ducts 104 are directly embedded within the body 96 such that the body 96 forms the walls of the cooling ducts 104, as opposed to self-contained cooling lines, apart from the body 96, that pass through the body 96. For example, in embodiments, the coolant is in contact with the body 96 as it travels through the cooling ducts 104. The body 96 of the RF coil 62 may be manufactured via an additive manufacturing process (e.g. three-dimensional printing) such that the cooling ducts 104 are formed as an integral part of the body 96. Alternatively, the cooling ducts 104 may be formed by drilling, etching, burning, lasing, evaporating, and/or other wise removing part of the material that forms the body 96.

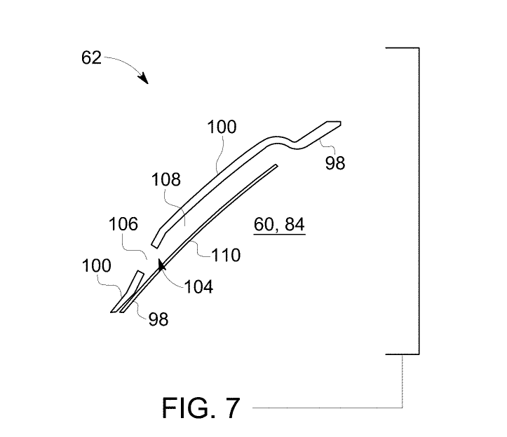

In embodiments, the cooling ducts 104 may run along the longitudinal axis 102 and/or circumferentially around the longitudinal axis 102. In embodiments, the cooling ducts 104 may include coolant intake openings 106 and/or coolant dispensing openings 108. The coolant intake openings 106 may be formed by the outer wall 100 of the body 96. For example, as can be seen in FIGS. 4 and 5, the coolant intake openings 106 may be flush with the body 96 so that the coolant may be drawn into (via a suction force) or pushed into (via a propelling force) the cooling ducts 104 such that the coolant flows from the coolant intake openings 106, through the cooling ducts 104, and out of the coolant displacement openings 108. The coolant dispensing opening 108 may be formed within the body 96 such that the directed coolant reaches assembly components that may be fully and/or partially contained/embedded within the RF coil 62, such as sensors and/or microprocessors. As best seen in FIG. 7, the coolant dispensing openings 108 may also be formed by the inner wall 98 and/or the outer wall 100. Additionally, the coolant intake openings 106 and the coolant dispensing openings 108 may be configured to direct coolant into the imaging bore 84 and/or imaging volume 60. In such embodiments, the coolant dispensing openings 108 may be hidden behind, flush with, and or otherwise obscured by one or more components in the imaging bore 84, such as a light panel 110.

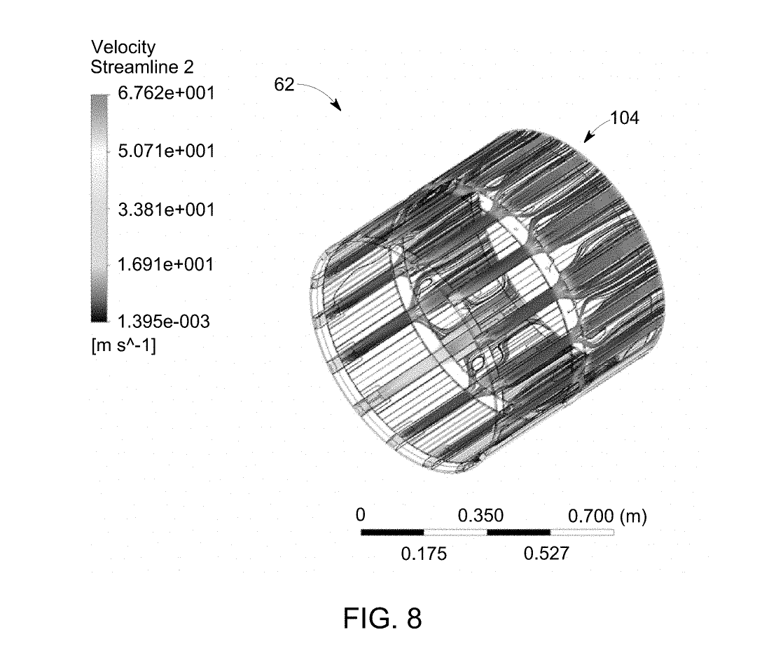

As illustrated in FIGS. 6 and 8, the cooling ducts 104 may be configured to locally manage the heat within the magnet assembly 56 and/or the encompassing MRI 10 by directing coolant to individual assembly components, other than the RF coil 62. In particular, the cooling ducts 104 may direct coolant to one or more assembly components (101 in FIG. 6) which may be embedded within the RF coil 62. Additionally, and as shown in FIG. 8, the cooling ducts 104 may also be configured to globally manage the heat within the magnet assembly 56 and/or the encompassing MRI 10 by directing coolant over, across, and/or through the RF coil 62 (which itself is an assembly component). For example, as shown in FIG. 8, the embedded cooling ducts 104 can efficiently direct coolant over and through the body 96 of the RF coil 62 such that the coolant is evenly distributed along the RF coil 62. Thus, the coolant can absorb and then remove a significant amount of heat from the RF coil 62 that may otherwise have been conducted towards the imaging bore 84.

Turning now to FIGS. 2 and 6, in embodiments, the RF coil 62 may include phase change material 112 disposed on the body 96 and/or fully and/or partially embedded within the body 96 between the inner 98 and outer 100 walls. In such embodiments, the phase change material 112 may be configured to absorb heat emitted by at least one assembly component of the magnet assembly 56 and/or the encompassing MRI 10, to include the RF coil 62. For example, heat generated by the gradient assembly 54 may be absorbed by the phase change material 112 before the RF coil 62 can conduct it to the imaging bore 84. Additionally, the phase change material 112 may absorb heat that has already made its way to the RF coil 62 and/or heat generated/emitted by the RF coil 62 itself. By absorbing the heat emitted from assembly components, the phase change material 112 extends the amount of time it takes for the temperature of the imaging core 84 to rise in response to the amount of heat emitted by the various assembly components of the magnet assembly 56 and/or the encompassing MRI 10. In other words, in embodiments, the phase change material 112 absorbs heat from one or more assembly components such that a rise in the temperature of the imaging bore 84 resulting from heat emitted by the one or more assembly component is delayed. As such, the phase change material 112 may be selected so as to have a phase transition temperature at or near the operating temperature of one or more assembly components. Additionally, the phase change material 112 may be of a bulk amount.

Further, while the embodiments depicted herein show the phase change material 112 embedded within the RF coil 62, it is to be understood that the phase change material 112 may be disposed on or embedded in other assembly components of the magnet assembly 56 and/or the encompassing MRI 10. Thus, the phase change material 112 may be configured to absorb heat from individual assembly components (i.e. localized heat management). Additionally, the phase change material 112 may also be configured to absorb heat that would normally have been absorbed/conducted by the RF coil 62 (i.e. globalized heat management).

Accordingly, embodiments of the present invention provide many benefits over traditional MRI systems. For example, in some embodiments, the cooling ducts 104 embedded directly into the body 96 of the RF coil 62 allow for the cooling of assembly components and/or the imaging bore 84 without the need for an additional cooling and/or shielding layer disposed within the magnet assembly 56. Thus, such embodiments are able to cool assembly components within a magnet assembly 56 without reducing the size of the imaging bore 84 and/or increasing the size of the magnet assembly 56. Moreover, in some embodiments, the RF coils 62 provides for a more uniform delivery of coolant over the RF coil 62 and/or other assembly components of a magnet assembly 56 and/or the encompassing MRI 10. Thus, in such embodiments, the embedded cooling ducts 104 may eliminate and/or reduce the effects of “dead spots,” which are regions of the RF coil 62 and/or other assembly components that do not receive adequate coolant. Further, by more efficiently distributing coolant within a RF coil 62, some embodiments reduce the amount of coolant needed to be supplied to RF coil 62, thereby allowing such embodiments to use smaller pumps and/or fans to propel the coolant through the cooling ducts 104.

Further, in some embodiments, the magnet assemblies 56 and/or the encompassing MRI 10 that include phase change material 112 embedded within and/or on a RF coil 62, or otherwise disposed within the magnet assembly 56 and/or the encompassing MRI 10, increase the heating time constant of the magnetic assembly 56 and/or the encompassing MRI 10, and in turn, extend the amount of operational/imaging time before the MRI system must be rested. For example, in some embodiments, the MRI systems 10 utilizes an appropriate type and/or amount of phase change material 112 in a RF coil 62 such that the MRI system may have a heating time constant ten (10) time or more than traditional MRI systems. Thus, such embodiments may increase the number and/or aggressiveness/quality of images that may be taken by a MRI system 10 within a given time period. Accordingly, such embodiments may further increase the efficiency and cost effectiveness of an MRI system 10.

It is to be understood that the above description is intended to be illustrative, and not restrictive. For example, the above-described embodiments (and/or aspects thereof) may be used in combination with each other. Additionally, many modifications may be made to adapt a particular situation or material to the teachings of the invention without departing from its scope.

For example, in an embodiment, a radio frequency coil is provided. The radio frequency coil includes a body and a cooling duct. The body has an inner wall and an outer wall opposite the inner wall. The body is configured to fit over an imaging bore of a magnetic resonance imaging system such that the inner wall is closer to the imaging bore than the outer wall. The cooling duct is embedded in the body between the inner wall and the outer wall and configured to direct a coolant to at least one assembly component disposed in the magnetic resonance imaging system. The cooling duct is formed by the body. In certain embodiments, the cooling duct includes at least one of a coolant intake opening formed by the outer wall and a coolant dispensing opening formed by the inner wall. In certain embodiments, the body is constructed via an additive manufacturing process. In certain embodiments, the cooling duct provides uniform distribution of the coolant to the at least one assembly component. In certain embodiments, the body has a longitudinal axis and the cooling duct runs at least one of circumferentially around the axis or longitudinally along the axis.

In another embodiment, another radio frequency coil is provided. The radio frequency coil includes a body and a phase change material. The body has an inner wall and an outer wall opposite the inner wall. The body is configured to fit over an imaging bore of a magnetic resonance imaging system such that the inner wall is closer to the imaging bore than the outer wall. The phase change material is configured to absorb heat emitted by at least one assembly component of the magnetic resonance imaging system. The phase change material is disposed on the body or embedded in the body between the inner wall and the outer wall. In certain embodiments, the at least one assembly component includes at least one of the radio frequency coil and a gradient coil. In certain embodiments, the phase change material has a phase transition temperature near an operating temperature of the at least one assembly component. In certain embodiments, the phase change material is a bulk amount. In certain embodiments, the bulk amount is sufficient to delay a rise in a temperature of the imaging bore resulting from heat emitted by the at least one assembly component.

In yet another embodiment, a method for managing heat is provided. The method includes cooling at least one assembly component of a magnetic resonance imaging system via a coolant directed by a cooling duct. The cooling duct is embedded between an inner wall and an outer wall of a body of a radio frequency coil. The outer wall is opposite the inner wall. The body is configured to fit over an imaging bore of a magnetic resonance imaging system such that the inner wall is closer to the imaging bore than the outer wall. The cooling duct is formed by the body. In certain embodiments, the at least one assembly component includes the radio frequency coil. In certain embodiments, the body is constructed via an additive manufacturing process. In certain embodiments, the body has a longitudinal axis and the cooling duct runs at least one of circumferentially around the axis or longitudinally along the axis.

In yet another embodiment, another method for managing heat is provided. The method includes absorbing, via a phase change material, heat emitted by at least one assembly component of a magnetic resonance imaging system. The phase change material is disposed on a body of a radio frequency coil or embedded in the body between an inner wall and an outer wall of the body. The outer wall is opposite the inner wall. The body is configured to fit over an imaging bore of the magnetic resonance imaging system such that the inner wall is closer to the imaging bore than the outer wall. In certain embodiments, the at least one assembly component includes the radio frequency coil. In certain embodiments, the phase change material has a phase transition temperature near an operating temperature of the at least one assembly component. In certain embodiments, the phase change material is a bulk amount. In certain embodiments the method further includes delaying a rise in a temperature of the imaging bore resulting from heat emitted by the at least one assembly component.

In yet another embodiment, a magnetic resonance imaging system is provided. The system includes at least one assembly component that emits heat, and an imaging bore. A bulk amount of a phase change material is disposed within the magnetic resonance imaging system. The phase change material has a phase transition temperature near an operating temperature of the at least one assembly component such that a rise in a temperature of the imaging bore resulting from heat emitted by at least one assembly component is delayed.

Additionally, while the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, terms such as “first,” “second,” “third,” “upper,” “lower,” “bottom,” “top,” etc. are used merely as labels, and are not intended to impose numerical or positional requirements on their objects. Further, the limitations of the following claims are not written in means-plus-function format are not intended to be interpreted based on 35 U.S.C. §112(f), unless and until such claim limitations expressly use the phrase “means for” followed by a statement of function void of further structure.

This written description uses examples to disclose several embodiments of the invention, including the best mode, and also to enable one of ordinary skill in the art to practice the embodiments of invention, including making and using any devices or systems and performing any incorporated methods. The patentable scope of the invention is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property.

Since certain changes may be made in the above-described invention, without departing from the spirit and scope of the invention herein involved, it is intended that all of the subject matter of the above description shown in the accompanying drawings shall be interpreted merely as examples illustrating the inventive concept herein and shall not be construed as limiting the invention.

What is claimed is:

1. A radio frequency coil comprising:
a body having an inner wall and an outer wall opposite the inner wall, the body configured to fit over an imaging bore of a magnetic resonance imaging system such that the inner wall is closer to the imaging bore than the outer wall;
a cooling duct embedded in the body between the inner wall and the outer wall and configured to direct a coolant to at least one assembly component disposed in the magnetic resonance imaging system; and
wherein the cooling duct is formed by the body.

2. The radio frequency coil of claim 1, wherein the cooling duct includes at least one of a coolant intake opening formed by the outer wall and a coolant dispensing opening formed by the inner wall.

3. The radio frequency coil of claim 1, wherein the body is constructed via an additive manufacturing process

4. The radio frequency coil of claim 1, wherein the cooling duct provides uniform distribution of the coolant to the at least one assembly component.

5. The radio frequency coil of claim 1, wherein the body has a longitudinal axis and the cooling duct runs at least one of circumferentially around the axis or longitudinally along the axis.

6. A radio frequency coil comprising:
A body having an inner wall and an outer wall opposite the inner wall, the body configured to fit over an imaging bore of a magnetic resonance imaging system such that the inner wall is closer to the imaging bore than the outer wall;
a phase change material configured to absorb heat emitted by at least one assembly component of the magnetic resonance imaging system; and
wherein the phase change material is disposed on the body or embedded in the body between the inner wall and the outer wall.

7. The radio frequency coil of claim 6, wherein the at least one assembly component includes at least one of the radio frequency coil and a gradient coil.

8. The radio frequency coil of claim 6, wherein the phase change material has a phase transition temperature near an operating temperature of the at least one assembly component.

9. The radio frequency coil of claim 6, wherein the phase change material is a bulk amount.

10. The radio frequency coil of claim 9, wherein the bulk amount is sufficient to delay a rise in a temperature of the imaging bore resulting from heat emitted by the at least one assembly component.

11. A method comprising:
cooling at least one assembly component of a magnetic resonance imaging system via a coolant directed by a cooling duct, the cooling duct embedded between an inner wall and an outer wall of a body of a radio frequency coil, the outer wall opposite the inner wall, the body configured to fit over an imaging bore of the magnetic resonance imaging system such that the inner wall is closer to the imaging bore than the outer wall; and
wherein the cooling duct is formed by the body.

12. The method of claim 11, wherein the at least one assembly component includes the radio frequency coil.

13. The method of claim 11, wherein the body is constructed via an additive manufacturing process.

14. The method of claim 11, wherein the body has a longitudinal axis and the cooling duct runs at least one of circumferentially around the axis or longitudinally along the axis.

15. A method comprising:
absorbing, via a phase change material, heat emitted by at least one assembly component of a magnetic resonance imaging system; and
wherein the phase change material is disposed on a body of a radio frequency coil or embedded in the body between an inner wall and an outer wall of the body, the outer wall opposite the inner wall, and the body configured to fit over an imaging bore of the magnetic resonance imaging system such that the inner wall is closer to the imaging bore than the outer wall.

16. The method of claim 15, wherein the at least one assembly component includes the radio frequency coil.

17. The method of claim 15, wherein the phase change material has a phase transition temperature near an operating temperature of the at least one assembly component.

18. The method of claim 15, wherein the phase change material is a bulk amount.

19. The method of claim 15, the method further comprising:
delaying a rise in a temperature of the imaging bore resulting from heat emitted by the at least one assembly component.

20. A magnetic resonance imaging system comprising:
at least one assembly component that emits heat;
an imaging bore; and
wherein a bulk amount of a phase change material is disposed within the magnetic resonance imaging system, the phase change material having a phase transition temperature near an operating temperature of the at least one assembly component such that a rise in a temperature of the imaging bore resulting from heat emitted by at least one assembly component is delayed.
